(19) United States
(12) Patent Application Publication
(10) Pub. No.: US 2025/0229789 A1
(43) Pub. Date: Jul. 17, 2025

(54) INFORMATION PROVISION SYSTEM, INFORMATION PROVISION CONTROL DEVICE AND INFORMATION PROVISION METHOD

(71) Applicant: DENSO CORPORATION, Kariya-city (JP)

(72) Inventors: Taro KOBAYASHI, Kariya-city (JP); Masafumi NOHARA, Kariya-city (JP); Keiji OKAMOTO, Kariya-city (JP); Kazuhiro MATSUI, Kariya-city (JP); Natsuko MIYAZAKI, Kariya-city (JP); Jinhae CHOI, Kariya-city (JP); Haozhou LI, Kariya-city (JP); Asuka SAKAI, Kariya-city (JP)

(21) Appl. No.: 19/096,801

(22) Filed: Apr. 1, 2025

Related U.S. Application Data
(62) Division of application No. 18/479,438, filed on Oct. 2, 2023.
Application No. 18/479,438 is a continuation of application No. PCT/JP2022/009287, filed on Mar. 4, 2022.

(30) Foreign Application Priority Data
Apr. 5, 2021 (JP) 2021-064275

(51) Int. Cl. B60W 40/09; B60W 40/08; B60W 50/14; G06V 20/59; G06V 40/18; G16H 40/67; G16H 80/00

(52) U.S. Cl. CPC B60W 40/09; B60W 50/14; G06V 20/597; G06V 40/18; G16H 40/67; G16H 80/00; B60W 2040/0827; B60W 2040/0872; B60W 2050/146; B60W 2420/403; B60W 2540/221; B60W 2540/225; B60W 2540/229; B60W 2556/10; B60W 2556/45

(57) ABSTRACT

An information provision system is configured to detect driver information, which is information including biological information of a driver, transmit information acquired inside the vehicle to a user terminal provided outside the vehicle, acquire the driver information, extract information necessary for grasping health condition of the driver from the driver information to generate health-related information, and transmit the health-related information to the user terminal.

### CROSS REFERENCE TO RELATED APPLICATION

This application is a divisional application of U.S. patent application Ser. No. 18/479,438 filed on Oct. 2, 2023 which is a continuation application of International Patent Application No. PCT/JP2022/009287 filed on Mar. 4, 2022, which designated the U.S. and claims the benefit of priority from Japanese Patent Application No. 2021-064275 filed on Apr. 5, 2021. The entire disclosure of all of the above applications are incorporated herein by reference.

### TECHNICAL FIELD

The present disclosure relates to an information provision system, an information provision control device, and an information provision method.

### BACKGROUND

JP2018-124791A describes an information provision system that provides information to a user who is driving a vehicle according to a combination of estimated emotion, driving skill, and physical condition. The disclosure of JP2018-124791A is incorporated herein by reference as an explanation of technical elements in the present disclosure.

### SUMMARY

The present disclosure describes an information provision system that is configured to detect driver information, which is information including biological information of a driver, transmit information acquired inside the vehicle to a user terminal provided outside the vehicle, acquire the driver information, extract information necessary for grasping health condition of the driver from the driver information to generate health-related information, and transmit the health-related information to the user terminal.

### BRIEF DESCRIPTION OF DRAWINGS

Objects, features and advantages of the present disclosure will become more apparent from the following detailed description made with reference to the accompanying drawings. In the drawings:

FIG. 1 is a block diagram relating to information provision control of an information provision system;

FIG. 2 is a block diagram showing the configuration of a driver information detection device;

FIG. 3 is a flowchart relating to information provision control of the information provision system;

FIG. 4 is a block diagram regarding information provision control of the information provision system in the second embodiment; and

FIG. 5 is a flow chart regarding information provision control of the information provision system in the second embodiment.

### DETAILED DESCRIPTION

In the configuration of a related art document, information is provided to the user who is driving the vehicle according to the combination of estimated emotion, driving skill and physical condition. The information provided to the user is information on driving guidance that is directly or indirectly related to driving. In the configuration of the related art, the driving guidance information generated based on the information obtained from the user is provided not to the outside but to the user himself/herself. It is not assumed to be provided to the outside of the vehicle. There is no disclosure of a configuration for externally providing and utilizing the information necessary for grasping the health condition of the driver of the vehicle. Further improvements may be required in the authentication system.

The present disclosure describes an information provision system or the like that can be used to improve the health of drivers.

According to one aspect of the present disclosure, an information provision system may comprise: a driver information detection device that is configured to detect driver information, which is information including biological information of a driver who drives a vehicle; a communication device that is configured to transmit information acquired inside the vehicle to a user terminal provided outside the vehicle; a driver information acquisition unit that is configured to acquire the driver information detected by the driver information detection device; an information generation unit that is configured to extract information necessary for grasping health condition of the driver from the driver information acquired by the driver information acquisition unit to generate health-related information; and an information communication unit that is configured to transmit the health-related information generated by the information generation unit from the communication device to the user terminal.

According to another aspect of the present disclosure, an information provision control device may comprise: a driver information acquisition unit that is configured to acquire the driver information detected by a driver information detection device which is configured to detect the driver information, which is information including biological information of a driver who drives a vehicle; an information generation unit that is configured to extract information necessary for grasping health condition of the driver from the driver information acquired by the driver information acquisition unit to generate health-related information; and an information communication unit that is configured to transmit the health-related information generated by the information generation unit from a communication device, which is configured to transmit information acquired inside the vehicle to a user terminal provided outside the vehicle, to the user terminal.

According to another aspect of the present disclosure, an information provision method may comprise: a driver information acquisition step that is configured to acquire the driver information detected by a driver information detection device which is configured to detect the driver information, which is information including biological information of a driver who drives a vehicle; an information generation step that is configured to extract information necessary for grasping health condition of the driver from the driver information acquired by the driver information acquisition step to generate health-related information; and an information communication step that is configured to transmit the health-related information generated by the information generation step from a communication device, which is configured to transmit information acquired inside the vehicle to a user terminal provided outside the vehicle, to the user terminal.

According to the information provision system or the like of the present disclosure, information necessary for grasping the health condition of the driver is extracted from the driver information, health-related information is generated, and the health-related information is transmitted to the user terminal. Therefore, it may be possible to generate the health-related information based on the driver information and to transmit it to the user terminal. Therefore, it may be possible that the user of the user terminal outside the vehicle obtains the driver's information and uses it to improve the driver's health. Therefore, it may be possible to provide an information provision system and the like that can be used to improve the health of drivers. In addition, the information generation unit extracts information necessary for grasping the health condition of the driver from the driver information acquired by the driver information acquisition unit, and generates health-related information. Therefore, it may be possible to reduce the data size of the information to be transmitted to the user terminal as compared to the case where all the acquired driver information is transmitted to the user terminal as it is.

The disclosed aspects in this specification adopt different technical solutions from each other in order to achieve their respective objectives.

Embodiments will be described with reference to drawings. In some embodiments, functionally and/or structurally corresponding and/or associated parts may be given the same reference numerals, or reference numerals with different digit placed on equal to or higher than a hundred place. For corresponding parts and/or associated parts, reference can be made to the description of other embodiments.

### First Embodiment

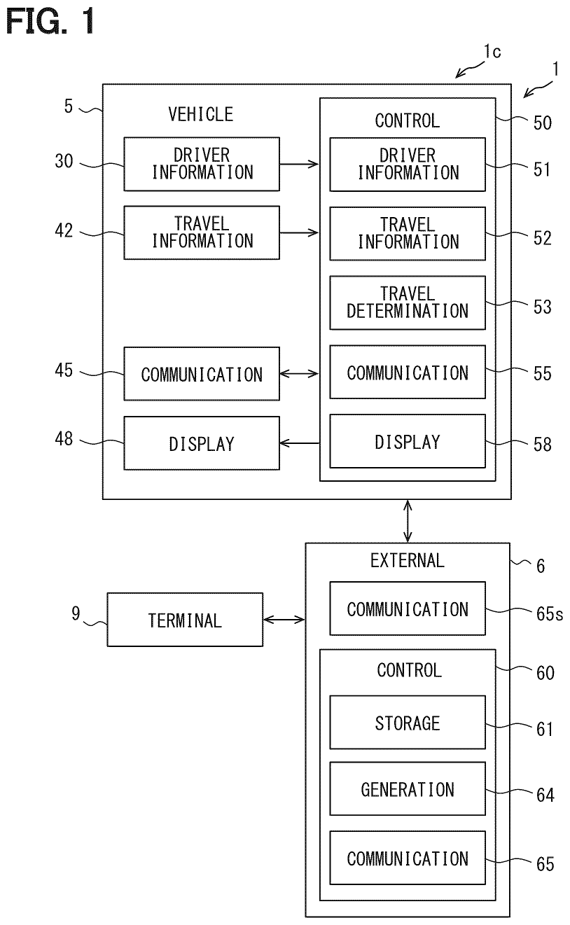

In FIG. 1, an information provision system 1 is a system for providing acquired information about the driver of a vehicle 5 to a user terminal 9, which is a terminal provided outside the vehicle 5. The information provision system 1 not only provides information on the driver of the vehicle 5 to the user terminal 9, but also provides the driver of the vehicle 5 with information transmitted from the user terminal 9. Therefore, the information provision system 1 is configured to allow bidirectional communication between the vehicle 5 and the user terminal 9. The information provision system 1 may be capable of providing at least information about the driver of the vehicle 5 to the user terminal 9. Therefore, it may be possible to employ a configuration in which the information transmitted from the user terminal 9 is not provided to the driver of the vehicle 5.

For example, the information provision system 1 may be utilized as a system for providing information necessary for remote diagnosis (may be referred to as a remote medical examination) by using the user terminal 9 as a terminal for electronic medical records in a medical institution. Alternatively, the information provision system 1 may be used as a system for providing information that can be used for health improvement or recovery from fatigue by using the user terminal 9 as a terminal of health equipment such as a massage chair at the driver's home. The utilization method of the information provision system 1 is not limited to the described method, and may be utilized in various scenes where the information acquired inside the vehicle is utilized outside the vehicle.

The information provision system 1 includes a vehicle 5 and an external server 6. The vehicle 5 includes a driver information detection device 30, a travel information detection device 42, a vehicle communication device 45 and a display device 48.

The travel information detection device 42 detects travel information, which is information relating to travel of the vehicle 5. The travel information detection device 42 includes multiple sensors that detect different information, such as a vehicle speed sensor that detects vehicle speed and a steering sensor that detects a steering angle. The travel information is associated with time information, and indicates how the vehicle 5 travels for each time.

The travel information includes information indicating whether the vehicle 5 is traveling or stopped. The travel information includes information indicating the operation results of the driver's brake, accelerator, steering, and the like. The travel information includes travel information such as sudden braking and sudden acceleration that may cause the driver to feel a great amount of stress momentarily. The travel information includes travel information that may cause the driver to continuously feel stress, such as driving at high speed or in a traffic jam.

The vehicle communication device 45 is a device for communicating with the external server 6. The vehicle communication device 45 transmits information from the vehicle 5 to the external server 6. The vehicle communication device 45 receives information transmitted from the external server 6 to the vehicle 5.

The display device 48 is a device for displaying information to the driver of the vehicle 5. The display device 48 may be a touch panel type display, and is configured to receive an input operation by the driver. The display device 48 may be, for example, a center information display provided substantially in the center of an instrument panel.

The driver information detection device 30 is a device for detecting driver information, which is information about the driver sitting in the driver's seat of the vehicle 5. The driver information includes biological information, which is information emitted by the driver who is a living organism. The driver information detection device 30 includes multiple sensors that detect different information. The driver information is associated with time information, and indicates the state of the driver for each time.

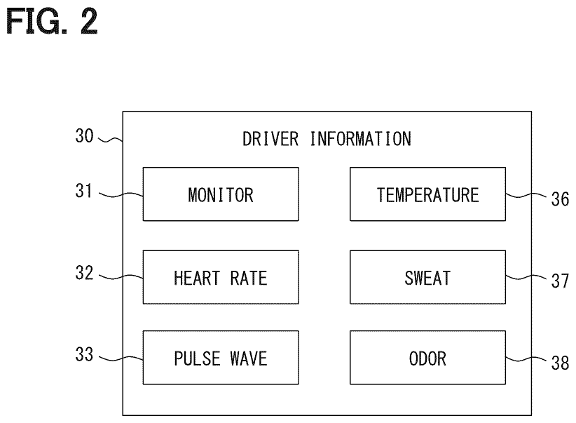

In FIG. 2, the driver information detection device 30 includes a driver monitoring device 31, a heart rate sensor 32, a pulse wave sensor 33, a body temperature sensor 36, a sweat sensor 37, and a body odor sensor. The driver information detection device 30 does not need to include all the devices described above. The driver information detection device 30 may include devices other than the devices and sensors described above.

The driver monitoring device 31 includes a near-infrared light source, a near-infrared camera, and a control unit for controlling them. The driver monitoring device 31 is arranged on the upper surface of the instrument panel with the near-infrared camera directed toward the driver's seat. The driver monitoring device 31 uses a near-infrared camera to capture an image of the driver's face irradiated with near-infrared light from a near-infrared light source. An image captured by the near-infrared camera is subjected to image analysis by the control unit. The control unit extracts, for example, the orientation of the driver's face and the degree of opening of the eyes from the captured image. The captured image captured using the driver monitoring device 31 may be used for face authentication of the driver.

The driver monitoring device 31 performs the face authentication of the driver and detects the face authentication result as driver information. The driver monitoring device 31 detects the facial expression of the driver as driver information. The driver monitoring device 31 detects the emotion and physical condition of the driver estimated from the facial expression of the driver.

The driver monitoring device 31 detects the line of sight of the driver as driver information. The driver monitoring device 31 detects, as driver information, the degree of concentration on driving estimated from changes in the driver's facial expression and line of sight.

The driver monitoring device 31 detects the driver's breathing speed as the driver information from the movement of the driver's mouth, nose, neck, and chest captured in the image. A microphone capable of acquiring sound from inside the vehicle may be provided to detect the driver's breathing speed by acquiring the sound of the driver's breathing.

The driver monitoring device 31 irradiates a specific visible light toward the driver, and captures the movement of the driver's eyes with respect to the visible light, thereby detecting the driver's pupil reflection as driver information. The pupil reflex detection is a detection that can only be performed when the vehicle 5 is not running, that is, when the vehicle 5 is parked. For example, the pupil reflex is performed only when the vehicle 5 is not running. The detection is not performed when the speed of vehicle 5 is not zero. The information detectable by the driver monitoring device 31 includes information detectable both while the vehicle 5 is running and parked, and information detectable only while the vehicle 5 is parked.

The driver monitoring device 31 monitors the driver by recognizing the speed of the vehicle 5, the driver's facial parts, the head posture, and the eye-opening degree. Face parts include eyes, nose, and mouth. The driver monitoring device 31 detects an inattentive state in which the driver looks aside, a drowsy state in which the driver feels sleepy, a dozing state in which the driver is dozing, and an inappropriate posture state in which the driver drives in an inappropriate driving posture. The inattentive state may be referred to as an abnormal face orientation in which the orientation of the face is not normal. The inappropriate posture state may be referred to as a face position abnormality in which the position of the face is not normal. A driver status monitor (registered trademark), for example, may be used as the driver monitoring device 31.

A heart rate sensor 32 is provided on the steering wheel. When the driver grips the portion of the steering wheel where the heart rate sensor 32 is provided, the heart rate sensor 32 detects the driver's heart rate as the driver information.

The pulse wave sensor 33, as similar to the heart rate sensor 32, is provided on the steering wheel. When the driver grips the portion of the steering wheel where the pulse wave sensor 33 is provided, the pulse wave sensor 33 detects contraction of the driver's blood vessels and detects the pulse wave as the driver information. The heart rate sensor 32 and the pulse wave sensor 33 may be configured to be detectable by one sensor.

The body temperature sensor 36 detects the body temperature of the driver as the driver information. As the body temperature sensor 36, for example, a non-contact thermometer which is able to detect body temperature in a non-contact manner may be employed. When the driver monitoring device 31 is able to detect temperature, the driver monitoring device 31 may also function as the body temperature sensor 36.

A sweat sensor 37 is provided on the steering wheel. When the driver grips the portion of the steering wheel where the sweat sensor 37 is provided, the sweat sensor 37 detects the amount of perspiration from the driver's hands as driver information.

The body odor sensor 38 detects the body odor of the driver as the driver information. The odor detected by the body odor sensor 38 is, for example, an odor called stress odor, which is generated when the driver is in a tense state or under strong stress. The odor detected by the body odor sensor 38 is a peculiar odor that occurs when the driver is sick, for example, the acetone odor that occurs in the case of diabetes. The body odor sensor 38 is preferably installed in the driver's seat or the like, which is in direct contact with the driver, so as not to misidentify the body odor of passengers other than the driver as the body odor of the driver.

Sensors such as the heart rate sensor 32 and the pulse wave sensor 33 that constitute the driver information detection device 30 are not limited to those provided on the steering wheel. For example, they may be provided in a seat such as a driver's seat. Alternatively, they may be a wearable terminal worn by the driver.

The driver information detection device 30 constantly detects the driver information such as body temperature and facial expression while the power switch of the vehicle 5 is in an on state. In other words, the driver information detection device 30 detects the driver information not only while the vehicle 5 is running, but also while the vehicle 5 is stopped or parked. For example, the driver information is detected even when the driver does not drive the vehicle 5 and is just sitting in the driver's seat, such as when the shift range of the vehicle 5 is in the parking range.

In FIG. 1, the vehicle 5 includes a vehicle control section 50 that controls information provision to the outside of the vehicle 5. The vehicle control unit 50 includes a driver information acquisition unit 51, a travel information acquisition unit 52, a travel determination unit 53, a vehicle communication unit 55 and a display control unit 58.

The driver information acquisition unit 51 acquires the driver information detected by the driver information detection device 30. The travel information acquisition unit 52 acquires the travel information detected by the travel information detection device 42. Both the driver information and the travel information are associated with the time information. Therefore, the acquired driver information can be associated with the travel information at the same timing.

Based on the travel information acquired by the travel information acquisition unit 52, the travel determination unit 53 determines whether it is in a normal state in which the vehicle is traveling normally or in an abnormal state in which the vehicle is traveling abnormally. The abnormal state includes a state in which the driver is performing a driving operation accompanied by sudden changes such as sudden braking, sudden acceleration, and sudden steering, or the like. Furthermore, the abnormal state includes a state to be in a traffic jam or a state to be driving at a high speed (for example, driving on an expressway). By contrast, the normal state is a state other than the abnormal state, and includes a state in which the vehicle 5 is parked or stopped. The traveling state is classified into either the normal state or the abnormal state. Thus, when a heart rate greatly deviating from a normal value is obtained as the driver information, it is determined whether or not the increase of the heart rate is caused by abnormal driving such as sudden braking, sudden acceleration, or the like.

The vehicle communication unit 55 uses the vehicle communication device 45 to transmit information to the outside of the vehicle 5, and receive information transmitted from the outside of the vehicle 5. The display control unit 58 controls display contents of the display device 48. The display control unit 58 displays, for example, medical examination results, which is described later.

The external server 4 is provided outside the vehicle 5. Information such as the driver information and the travel information transmitted from the vehicle 5 is stored in the external server 6. For example, a server provided on the cloud may be used as the external server 6.

The external server 6 includes a server communication device 65s and a server control unit 60. The server communication device 65s is a device for communicating with the vehicle 5 and the user terminal 9. The vehicle communication device 45 is able to transmit information to the vehicle 5 and the user terminal 9. The server communication device 65s is able to receive information transmitted from the vehicle 5 or the user terminal 9 to the external server 6. The server communication device 65s provides an example of a communication device.

The server control unit 60 controls information provision in the external server 6. The server control unit 60 includes an information storage unit 61, an information generation unit 64 and a server communication unit 65. The information storage unit 61 stores information received by the external server 6 and information generated by the external server 6. The information storage unit 61 stores not only the latest information received by the external server 6 but also information received in the past.

The information generation unit 64 generates health-related information to be transmitted to the user terminal 9 from the driver information received by the external server 6. The health-related information is information for estimating the health condition of the driver. Details of the health-related information is described later.

The server communication unit 65 communicates between the external server 6 and the vehicle 5, enabling bidirectional transmission and reception. The server communication unit 65 performs communication between the external server 6 and the user terminal 9, enabling bidirectional transmission and reception. The server communication unit 65 provides an example of an information communication unit.

The vehicle control unit 50 and the server control unit 60 constitute an information provision control device 1c. The information provision control device 1c is a device responsible for a series of controls in the information provision system 1.

The user terminal 9 is a device that receives information transmitted from the external server 6 and enables a user outside the vehicle 5 to use information about the driver of the vehicle 5. The user terminal 9 is, for example, an electronic medical record device provided in a medical institution. In this case, the user of the user terminal 9 is a doctor. The doctor may use the user terminal 9 to examine the driver.

Information provision control using the information provision system 1 will be described below. An example will be described in which remote medical examination is performed in which the driver who rides on the vehicle 5 is examined by a medical institution outside the vehicle 5.

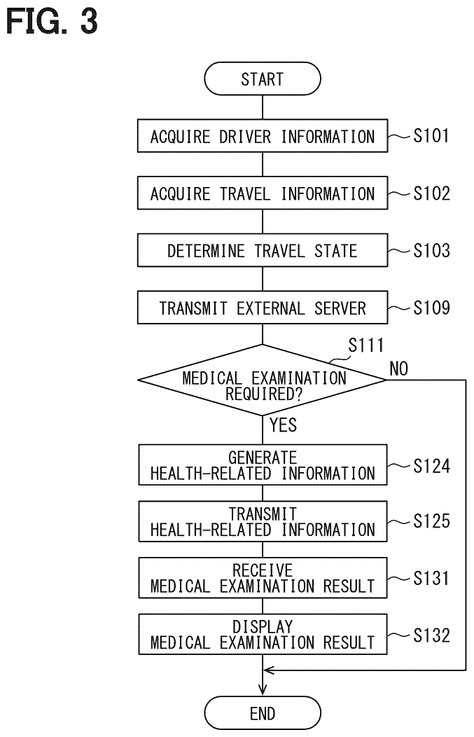

In FIG. 3, when information provision control is started by turning on the power switch of the vehicle 5, the driver information acquisition unit 51 acquires the driver information in S101. While the power switch of the vehicle 5 is on, that is, is in an on state, the driver information detection device 30 continues to detect the driver information. The driver information acquisition unit 51 acquires the latest driver information detected by the driver information detection device 30. After acquiring the driver information, the process proceeds to S102. S101 provides an example of a driver information acquisition step.

In S102, the travel information acquisition unit 52 acquires travel information. While the power switch of the vehicle 5 is on, the travel information detection device 42 continues to detect the travel information. The travel information acquisition unit 52 acquires the latest travel information detected by the travel information detection device 42. After acquiring the travel information, the process proceeds to S103. S102 may be referred to as a travel information acquisition step.

In S103, the travel determination unit 53 determines whether or not the traveling state of the vehicle 5 is in the normal state or in the abnormal state. The determination result as to whether it is in the normal state or the abnormal state constitutes part of the travel information. After determining whether the traveling state is in the normal state or in the abnormal state, the process proceeds to S109. S103 may be referred to as a traveling state determination step.

In S109, the vehicle communication unit 55 transmits the driver information and the travel information to the external server 6. The vehicle communication unit 55 transmits the acquired information to the external server 6 each time the vehicle communication unit 55 acquires the driver information and the travel information. Accordingly, the information about the driver is transmitted to the external server 6 in real time.

The vehicle communication unit 55 is not limited to a configuration that performs transmission to the external server 6 in real time. For example, the configuration may be such that the driver information and the travel information are transmitted each time a preset transmission interval elapses. In this case, the driver information and the travel information acquired over multiple timings are collectively transmitted in one transmission.

Each piece of information transmitted from the vehicle communication unit 55 is stored in the external server 6 by the information storage unit 61. The external server 6 stores multiple pieces of driver information and travel information detected at different timings. After transmitting the driver information and the travel information to the external server 6, the process proceeds to S111.

In S111, the vehicle control unit 50 determines whether or not a medical examination by a doctor is necessary. For example, when the driver presses a medical examination start button displayed on the center information display, it is determined that medical examination is necessary. When the medical examination is necessary (S111: YES), the process proceeds to S124. By contrast, when the medical examination is not necessary (S111: NO), it is determined that there is no need to transmit information to the user terminal 9, and the information provision control terminates. It may be preferable to restart the information provision control after ending the information provision control. Accordingly, the control of acquiring the driver information and storing it in the external server 6 can be continued regardless of whether or not the remote medical examination is required.

In S124, the information generation unit 64 generates diagnostic information as the health-related information based on the driver information. The diagnosis information, which is a type of the health-related information, is information necessary for a doctor to grasp the health condition of the driver and perform an examination. The health-related information includes the driver information detected while the vehicle 5 is not running.

The information generation unit 64 generates the diagnostic information by extracting specific information from the driver information. The diagnostic information includes, for example, information of heart rate, pulse wave, body temperature, breathing speed, pupillary reflex, line of sight, facial expression, amount of hand sweat, or body odor. The driver information that has little or no dependence on the driver's health status is not included in the diagnostic information. The information that is not included in the diagnostic information is, for example, information that detects an abnormal face orientation or an abnormal face position. The diagnostic information may be information that is not directly related to the driving operation of the vehicle 5 and may include information related to the driver's health. The health-related information that is not directly related to the driving operation is information that does not affect whether to stop driving immediately.

The information generation unit 64 does not include in the diagnostic information the driver information during the period when the travel determination unit 53 determines to be the abnormal state. For example, the information such as information of the heart rate at the moment of sudden braking is not included in the diagnostic information. This is because it is expected that the heart rate will be higher than normal due to sudden braking.

In addition, when the brake is applied suddenly, it is assumed that the heart rate may continue to be high not only at the moment the brake is applied but also for a while after that. For this reason, the driver information during the period from the end of the period determined to be the abnormal state to the elapse of the predetermined grace period is not included in the diagnosis information. In other words, the diagnostic information includes only the driver information when the driver is driving calmly or when the vehicle is parked or stopped without running.

The information generation unit 64 generates the diagnosis information based on the driver information and the travel information that have been transmitted from the start of detection of the driver information to the present. In other words, the diagnostic information can be generated using the driver information that is continuously acquired while the driver is in the vehicle. For example, it is assumed that thirty minutes have elapsed from the start of driver information detection to the present. In this case, the diagnostic information is generated based on the driver information and travel information acquired and transmitted within thirty minutes. According to this, the average values of heart rate and body temperature can be calculated with high accuracy. It is also possible to calculate changes in heart rate and body temperature over time. After generating the diagnostic information, the process proceeds to S125. S124 provides an example of an information generation step.

In S125, the server communication unit 65 transmits the diagnostic information to the user terminal 9. In the transmission of the diagnostic information, all generated diagnostic information such as heart rate, pulse wave, body temperature and the like are collectively transmitted. By contrast, the diagnostic information may be divided into a plurality of pieces and transmitted. After transmitting the diagnostic information, the process proceeds to S131. S125 provides an example of an information communication step.

By transmitting the diagnostic information to the user terminal 9 via the external server 6, the doctor can use the user terminal 9 to check the diagnostic information. For example, if the diagnostic information includes information on changes in body temperature over time, the doctor can consider the changes in body temperature over time in the diagnosis. Even if the body temperature changes greatly over time, it is possible to grasp the temperature when the body temperature is the highest and the temperature when the body temperature is the lowest.

In addition, the doctor can check not only the latest diagnostic information newly transmitted this time but also the diagnostic information transmitted in the past. By comparing the current body temperature with the body temperature when the driver is in a healthy state, it may be possible to easily grasp how much the driver is in a different state than when the driver is in the healthy state. For example, if the driver's normal temperature is lower than the general normal temperature, the current body temperature can be assessed and examined, taking into account the driver's lower normal temperature.

In S131, the vehicle communication unit 55 uses the vehicle communication device 45 to receive a medical examination result. The medical examination result is the result of medical examination performed by a doctor based on the diagnostic information transmitted to the user terminal 9. The medical examination result is transmitted from the user terminal 9 to the external server 6 and then transmitted from the external server 6 to the vehicle communication device 45 of the vehicle 5. After receiving the medical examination result, the process proceeds to S132.

In S132, the display control unit 58 displays the examination result on the display device 48. As a result, the driver can check the medical examination result by the doctor and grasp his/her own health status. As the medical examination result, the name of the disease, effective over-the-counter medications, a message recommending a blood test that cannot be performed by remote diagnosis are displayed, as necessary. After displaying the medical examination results, the information provision control terminates.

Information provision control using the information provision system 1 enables remote diagnosis by providing information necessary for medical examination by a doctor. Such a remote medical examination allows a doctor to examine a driver without a face-to-face meeting between the doctor who conducts medical examination and the driver to be examined, and improves the health of the driver in a simple and convenient manner. In addition, since the chances of direct contact between the doctor and the driver can be reduced, it may be possible to reduce the risk of spreading infectious diseases.

A case where the vehicle communication device 45 mounted on the vehicle 5 receives the medical examination results is described as an example. The device that receives the medical examination results is not limited to the vehicle communication device 45. For example, a mobile terminal of the driver may be configured to receive the medical examination results. In this case, the received medical examination result can be displayed on the screen of the mobile terminal.

According to the embodiment, the information provision system 1 includes the information generation unit 64 that extracts the information necessary for grasping the health condition of the driver from the driver information acquired by the driver information acquisition unit 51 and generates the health-related information. Furthermore, the information provision system 1 includes a server communication unit 65 that transmits the health-related information generated by the information generation unit 64 from the server communication device 65s to the user terminal 9. Therefore, the information provision system 1 is able to generate the health-related information of the driver and transmit it to the user terminal 9. Therefore, it may be possible that the user of the user terminal 9 utilizes the driver's health-related information. Therefore, it may be possible to provide an information provision system and the like that can be used to improve the health of the driver.

The information generation unit 64 extracts the information necessary for grasping the health condition of the driver from the driver information acquired by the driver information acquisition unit 51, and generates the health-related information. Therefore, it may be possible to reduce the data size of the information to be transmitted to the user terminal 9 as compared to the case where all the acquired driver information is transmitted to the user terminal 9 as it is.

The driver information acquisition unit 51 includes the driver information detected while the vehicle 5 is not running, in the driver information to be acquired. Therefore, compared to the case where the driver information acquisition unit 51 acquires only the driver information while the vehicle 5 is running, more driver information can be acquired. In addition, it may be assumed that the driver is relaxed and at rest when the vehicle 5 is not running compared to when the vehicle 5 is running. Therefore, it may be possible to reduce the influence of the traveling of the vehicle 5 on the acquired driver information.

The information generation unit 64 does not include the driver information in the health-related information during the period when the travel determination unit 53 determines to be the abnormal state. The driver information when an abnormality in the heart rate occurs due to abnormal driving such as sudden braking or high-speed driving is not used as the diagnostic information, which is a type of the health-related information. Therefore, it may be possible to suppress temporary changes in biological information due to the abnormal driving from being included in the diagnostic information.

The information generation unit 64 does not include in the health-related information the driver information detected during the period between the end of the period when the travel determination unit 53 determines to be the abnormal state and the elapse of the grace period. Therefore, the driver information immediately after an abnormality such as heart rate occurs due to abnormal driving, such as sudden braking or high-speed driving, is not used as the diagnostic information, which is a type of the health-related information. Therefore, it may be possible to suppress the inclusion of temporary changes in biological information due to the abnormal driving in the diagnostic information.

The travel determination unit 53 determines that a period during which the vehicle 5 is rapidly accelerating or decelerating is in the abnormal state. Therefore, even if the driver is significantly affected by sudden acceleration or deceleration, it may be possible to prevent the diagnostic information, which is a type of the health-related information, from being affected by sudden acceleration or deceleration.

The information generation unit 64 generates, as the health-related information, the diagnostic information that is used for medical examination by a doctor. Therefore, the information provision system 1 can be used for remote diagnosis by a doctor.

The display control unit 58 displays the result of the medical examination on the display device 48. Therefore, it may be possible to check the medical examination result by remote medical examination in the vehicle. Therefore, when information necessary for the medical examination is lacking, it is easy to immediately add information using the driver information detection device 30 mounted on the vehicle 5.

The information provision control device 1c includes an information generation unit 64 that extracts information necessary for grasping the health condition of the driver from the driver information acquired by the driver information acquisition unit 51 to generate the health-related information. Furthermore, the information provision control device 1c includes a server communication unit 65 that transmits the health-related information generated by the information generation unit 64 from the server communication device 65s to the user terminal 9. Therefore, the information provision control device 1c is able to generate the health-related information of the driver and transmit it to the user terminal 9. Therefore, the user of the user terminal 9 can utilize the driver's health-related information. Therefore, it may be possible to provide an information provision control device 1c that can be used to improve the health of drivers.

The information generation unit 64 of the information provision control device 1c extracts the information necessary for grasping the health condition of the driver from the driver information acquired by the driver information acquisition unit 51 to generate the health-related information. Therefore, it may be possible to reduce the data size of the information to be transmitted to the user terminal as compared to the case where all the acquired driver information is transmitted to the user terminal as it is.

The information provision method includes an information generation step that extracts information necessary for grasping the health condition of the driver from the driver information acquired by the driver information acquisition step and generates the health-related information. Further, the information provision method includes an information communication step that transmits the health-related information generated in the information generation step from the server communication device 65s to the user terminal 9 provided outside the vehicle 5. Therefore, it may be possible to generate the health-related information and transmit it to the user terminal 9. Therefore, it may be possible that the user of the user terminal 9 utilizes the driver's health-related information. Therefore, it may be possible to provide an information provision method that can be used to improve the health of drivers.

The information generation step extracts information necessary for grasping the health condition of the driver from the driver information acquired by the driver information acquisition step, and generates health-related information. Therefore, it may be possible to reduce the data size of the information to be transmitted to the user terminal as compared to the case where all the acquired driver information is transmitted to the user terminal as it is.

### Second Embodiment

The present embodiment is a modification of the above-described embodiments. In this embodiment, the vehicle control unit 50 includes an information generation unit 254 that generates the health-related information. The information generation unit 254 also generates fatigue level information, which is information indicating the driver's fatigue level, as the health-related information.

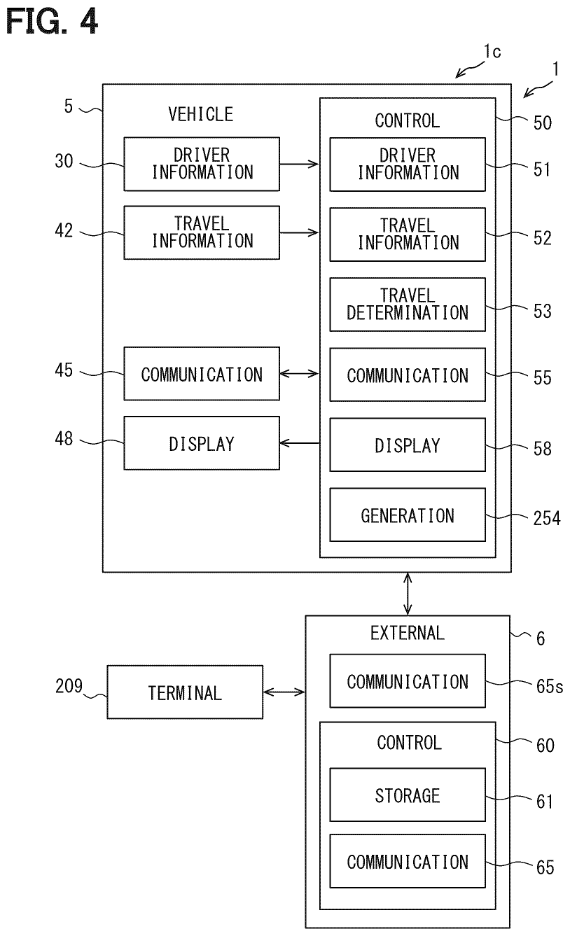

In FIG. 4, the vehicle control unit 50 includes the information generation unit 254. The information generation unit 254 generates the health-related information from the driver information acquired by the driver information acquisition unit 51. The information generation unit 254 generates, for example, the fatigue level information as the health-related information. The details of the fatigue level information will be described later.

The user terminal 9 is a device that receives information transmitted from the external server 6 and enables a user outside the vehicle 5 to use information about the driver of the vehicle 5. The user terminal 209 is, for example, a massage chair that the driver uses at home. In this case, the user of the user terminal 209 is the driver who has finished driving the vehicle 5.

The following will describe an example of information provision control using the information provision system 1. The following is an example of changing the settings of a massage chair placed outside the vehicle 5 according to the fatigue level of the driver.

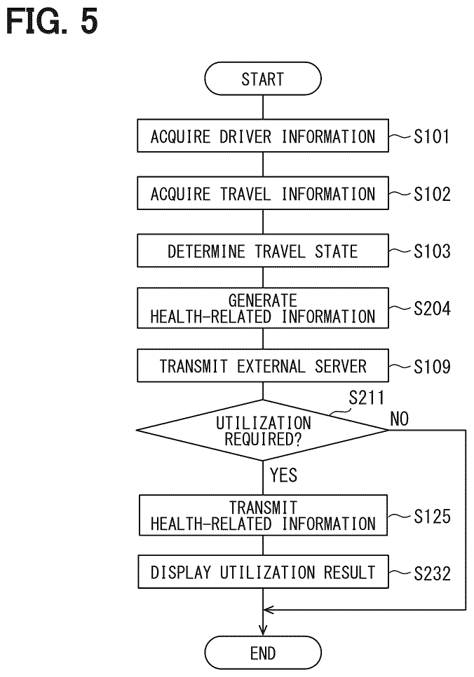

In FIG. 5, when the information provision control is started by turning on the power switch of the vehicle 5, the driver information acquisition unit 51 acquires the driver information in S101, and then proceeds to S102. In S102, the travel information acquisition unit 52 acquires travel information, and then proceeds to S103. In S103, the travel determination unit 53 determines whether or not the traveling state of the vehicle 5 is in the normal state or in the abnormal state, and then proceeds to S204.

In S204, the information generation unit 254 generates the fatigue level information as the health-related information, based on the driver information. The fatigue level information, which is a type of the health-related information, is information indicating the degree of fatigue in each part of the driver's body, such as arms, legs, and neck.

The information generation unit 254 generates the fatigue level information by extracting and analyzing specific information from the driver information. The degree of fatigue of the driver can be estimated from the captured image of the driver captured using the driver monitoring device 31. More specifically, the overall fatigue level is estimated from the driver's expression, complexion, heart rate, body temperature, and the like. For example, when the driver's facial expression or complexion is not good, it may be estimated that the overall degree of fatigue is high. Alternatively, when the driver's heart rate is higher than a normal value or the body temperature is higher than normal, it may be estimated that the driver's overall fatigue level is high.

Also, the degree of fatigue of each part is estimated from the line of sight and movement of the driver. For example, the more often a driver looks at his or her arms or legs, the higher the fatigue level in the area where his eyes are turned can be estimated. Alternatively, when the driver is making an action that worries the neck, such as rubbing the neck, it can be estimated that the degree of neck fatigue is high. The driver information that has little or no dependence on the driver's fatigue is not included in the fatigue level information. The information that is not included in the fatigue level information is, for example, information obtained by detecting pupillary reflex and the amount of hand sweat.

The information generation unit 254 does not include the driver information during the period when the travel determination unit 53 determines to be in an abnormal state in the fatigue level information. For example, the information about the line of sight at the moment of sudden braking is not included in the fatigue level information. This is to prevent an erroneous analysis of a change in the line of sight due to sudden braking as a change in the line of sight due to poor physical condition or fatigue.

In addition, when the brake is applied suddenly, it is assumed that the line-of-sight changes frequently not only at the moment when the brake is applied suddenly but also for a while after that. For this reason, the driver information during the period from the end of the period determined to be the abnormal state to the elapse of the predetermined grace period is not included in the fatigue level information. In other words, the fatigue level information includes only the driver information when the driver is driving calmly or when the vehicle is parked or stopped without running.

The information generation unit 254 generates the fatigue level information based on the travel information and the driver information that has been transmitted from the start of detection of the driver information to the present. In other words, the fatigue level information can be generated using the driver information that is continuously acquired while the driver is in the vehicle. For example, when thirty minutes have elapsed from the start of driver information detection to the present, the fatigue level information is generated based on the driver information and the travel information acquired during the thirty minutes. Accordingly, it may be possible to detect, for example, a change in line of sight or the like for thirty minutes and generate a fatigue level with high accuracy. After generating the fatigue level information, the process proceeds to S109. S204 provides an example of an information generation step.

In S109, the vehicle communication unit 55 transmits the fatigue level information, which is the health-related information, to the external server 6. The vehicle communication unit 55 transmits the generated fatigue level information to the external server 6 every time the fatigue level information is generated based on the driver information and the travel information. Accordingly, the information about the driver is transmitted to the external server 6 in real time. However, the fatigue level information generated from when the power switch of the vehicle 5 is turned on until it is turned off may be sent all at once immediately after the power switch of the vehicle 5 is turned off.

The fatigue level information transmitted from the vehicle communication unit 55 is stored in the external server 6 by the information storage unit 61. Therefore, the external server 6 stores the fatigue level information generated at different timings, such as the fatigue level information generated in the previous driving and the fatigue level information generated in the current driving. After transmitting the fatigue level information to the external server 6, the process proceeds to S211.

In S211, the vehicle control unit 50 determines whether or not the user terminal 209 needs to utilize the fatigue level information. More specifically, when the driver presses a utilization start button displayed on the center information display, it is determined that utilization of the fatigue level information is necessary. When it is necessary to utilize it (S211: YES), the process proceeds to S125. By contrast, when it is unnecessary to utilize it (S211: NO), it is determined that there is no need to transmit information to the user terminal 209, and the information provision control terminates. It may be preferable to restart the information provision control after ending the information provision control. As a result, the fatigue level information can be acquired and stored in the external server 6 regardless of whether or not the fatigue level information is to be used.

In S125, the server communication unit 65 transmits the fatigue level information to the user terminal 209, and then proceeds to S232. By transmitting the fatigue level information to the user terminal 209 via the external server 6, the settings of the massage chair, which is the user terminal 209, can be changed. For example, when the overall fatigue level is higher than normal, massage time may be set longer. Alternatively, when the arm is more fatigued than other parts, arm massage may be set to be strong.

In S232, the display control unit 58 displays the utilization result. More specifically, the setting of the massage chair changed based on the fatigue level information is displayed on the display device 48. For example, it displays that it has been determined that the fatigue level of the arm is high and that the massage for the arm has been changed from light operation to strong operation. After displaying the utilization result, the information display control is terminated.

According to the embodiment, the information generation unit 254 generates the fatigue level information indicating the driver's fatigue level as the health-related information. Therefore, the fatigue level information can be utilized outside the vehicle 5. For example, by using it to change the settings of a massage chair, it is possible to promote the driver's recovery from fatigue using the massage chair and improve the driver's health.

### Other Embodiments

The disclosure in this specification and drawings is not limited to the exemplified embodiments. The present disclosure includes embodiments described above and modifications of the above-described embodiments made by a person skilled in the art. For example, the disclosure is not limited to components and/or combinations of elements presented in the embodiments provided herein. The present disclosure may be implemented in various combinations thereof. The disclosure may have additional components that can be added to the embodiments. The present disclosure also includes modifications which include partial components/elements of the above-described embodiments. The present disclosure also includes replacement or combination of components and/or elements between one embodiment and another. The technical scope disclosed in the present disclosure is not limited to the above-described embodiments. It should be understood that some disclosed technical scopes are indicated by description of claims, and includes every modification within the equivalent scope and the scope of description of claims.

The disclosure in the specification, drawings and the like are not limited by the description of the claims. The disclosures in the specification, the drawings, and the like include the technical ideas described in the claims, and further extend to a wider variety of technical ideas than those described in the claims. Therefore, various technical ideas can be extracted from the disclosure of the specification, the drawings and the like without being limited to the description of the claims.

The control unit and method thereof according to the present disclosure may be implemented by one or more special-purposed computers. Such a special- purposed computer may be provided (i) by configuring (a) a processor and a memory programmed to execute one or more functions embodied by a computer program, or (ii) by configuring (b) a processor including one or more dedicated hardware logic circuits, or (iii) by configuring by a combination of (a) a processor and a memory programmed to execute one or more functions embodied by a computer program and (b) a processor including one or more dedicated hardware logic circuits. The technique for realizing the functions of each functional unit included in the apparatus or the method thereof does not necessarily need to include software, and all the functions may be realized using one or more hardware circuits. The computer program may be stored in a computer-readable non-transition tangible recording medium as an instruction executed by a computer.

What is claimed is:

1. An information provision system comprising:
a driver information detection device that is configured to detect driver information, which is information including biological information of a driver who drives a vehicle;
a communication device that is configured to transmit information acquired inside the vehicle to a user terminal provided outside the vehicle;
a driver information acquisition unit that is configured to acquire the driver information detected by the driver information detection device;
an information generation unit that is configured to extract information necessary for grasping health condition of the driver from the driver information acquired by the driver information acquisition unit to generate health-related information; and
an information communication unit that is configured to transmit the health-related information generated by the information generation unit from the communication device to the user terminal,
wherein
the information generation unit generates, as the health-related information, diagnostic information that is used for medical examination by a doctor, and
the communication device transmits a latest diagnostic information and a diagnostic information including information on changes in body temperature over time transmitted in past to the doctor using the user terminal via an external server.

2. The information provision system according to claim 1, further comprising:
a display device that is configured to display information to the driver; and
a display control unit that is configured to control display content displayed on the display device,
wherein
the communication device receives a result of the medical examination by the doctor who has used the diagnostic information, and
the display control unit displays the result of the medical examination on the display device.
